(19) United States
(12) Patent Application Publication
(10) Pub. No.: US 2010/0233231 A1
(43) Pub. Date: Sep. 16, 2010

(54) USE OF CRYOGENIC PROCESSING TO OBTAIN A SUBSTANTIALLY-THICKENED FORMULATION

(76) Inventors: Roger LABRECQUE, Londonderry, NH (US); Suzanne CONROY, Dracut, MA (US); Scott E. CORBEIL, Litchfield, NH (US); Paul MARTAKOS, Pelham, NH (US)

Correspondence Address: LAHIVE & COCKFIELD, LLP;FLOOR 30, SUITE 3000, ONE POST OFFICE SQUARE, BOSTON, MA, 02109, US

(21) Appl. No.: 12/401,228

(22) Filed: Mar. 10, 2009

(51) Int. Cl. A61F 2/00; A61K 31/436; A61K 31/355; A61K 31/70; A61K 31/337; A61K 9/14; A61K 38/13

(52) U.S. Cl. 424/423; 514/291; 514/458; 514/23; 514/449; 424/489; 514/11

(57) ABSTRACT

The present invention related to substantially-thickened therapeutic formulation comprising an oil-based composition and a therapeutic agent, wherein the therapeutic agent is of a reduced particle size.

### BACKGROUND OF THE INVENTION

Pharmaceutical drugs often have a highly polydisperse particle size distribution. Drug forms that are too large can negatively impact bioavailability, processing and manufacturability of products that use the drug in particulate form. A reduction in particle size is a common method of overcoming these problems.

For example, the rate of dissolution of a particulate drug can increase with specific surface area, e.g., with decreasing particle size. This rate of dissolution increase can result in enhanced bioavailability of the particulate drug. Particle size reduction is also useful in sustained release compositions in which a drug is dispersed within a matrix, for example, a polymer matrix or a gel matrix. Particle size reduction may control or modulate the initial release or burst often associated with sustained release compositions.

Many techniques are known in the art for reducing particle size, e.g., spray-drying, extrusion spheronization, spray-chilling, emulsion solvent evaporation/extraction, recrystallization, air jet milling and use of super critical fluids. However, all existing techniques suffer from various drawbacks. Most often these particle size reduction techniques are impractical because the techniques impart harmful heat to the particles. Pharmaceutical products are generally heat sensitive and degrade in the presence of excessive heat. Thus, under traditional particle size reduction techniques, the integrity of a pharmaceutical product may be compromised.

Once formed, the plurality of particles can be used in numerous ways, in vivo. For example, the particles can be used in a dry powder in inhaler treatments, an aqueous dispersion for intravenous use, or a capsule for oral delivery. The particles can also be applied topically, incorporated into coatings or directly into devices. The particles can also be sprinkled over a desired location in vivo.

However, a more controlled delivery, i.e., localized delivery, of the particles is also often desired. A composition that allows for the precise placement of a pharmaceutical to a target (e.g., an area of injury) is desired. A substantially-thickened formulation for application in vivo is one way to achieve this.

### SUMMARY OF THE INVENTION

Described herein is a method for forming a substantially-thickened formulation comprising at least one dispersing media and a dispersed agent, for example, a therapeutic agent, that can be used in vivo. Various embodiments of the substantially-thickened formulation can be used as coatings for implantable devices, e.g., stents, grafts, adhesion barriers, mesh, slings, prostheses, etc., as well as for improved sustained release formulations for the therapeutic agents contained therein.

To form the substantially-thickened formulations of the invention, a cryogenic liquid is used to bring compositions comprising at least one dispersing media and a solid therapeutic agent to a cryogenic temperature. The cooled composition is then fragmented, returned to room temperature and optionally sheared, resulting in a substantially-thickened formulation that can be used, for example, as a coating for implantable devices, e.g., stents, as well as for a sustained release formulation for the therapeutic agent contained therein.

Thus, in one aspect, the invention provides a substantially-thickened therapeutic formulation comprising an oil-based composition and a therapeutic agent, wherein the therapeutic agent is of a reduced particle size. In one embodiment, the particle size of the therapeutic agent has been reduced using cryogrinding (e.g., using a cryomill). In one embodiment, the therapeutic agent is at a loading concentration that is higher than the maximum solubility concentration of the agent in the oil-based composition before cryogrinding.

This process is advantageous, because the maximum loading of the therapeutic agent in the oil-based composition is higher than the maximum solubility before the therapeutic agent was cryoground. Thus, in one embodiment, the therapeutic agent is at a loading concentration of greater than 30% when the solubility of the agent in the oil-based composition before cryogrinding is less than 25%. In another embodiment, the therapeutic agent is at a loading concentration of 20%-50%.

In another embodiment, the therapeutic agent of the substantially-thickened formulation is uniformly dispersed throughout the formulation, such that there is no bulk phase separation between the oil-based composition and the therapeutic agent.

In one embodiment of the formulation, the oil-based composition comprises an oil containing at least one lipid or fatty acid. In another embodiment, the fatty acid comprises one or more of arachidic acid, gadoleic acid, arachidonic acid, eicosapentaenoic acid (EPA), docosahexaenoic acid (DHA), butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, analogs and pharmaceutically acceptable salts thereof. In another embodiment, the oil-based composition comprises at least one or more of a fish oil, a biological oil, or vegetable oil.

The oil-based composition can include a vitamin E compound, such as alpha-tocopherol, beta-tocopherol, delta-tocopherol, gamma-tocopherol, alpha-tocotrienol, beta-tocotrienol, delta-tocotrienol, gamma-tocotrienol, alpha-tocopherol acetate, beta-tocopherol acetate, gamma-tocopherol acetate, delta-tocopherol acetate, alpha-tocotrienol acetate, beta-tocotrienol acetate, delta-tocotrienol acetate, gamma-tocopherol acetate, alpha-tocopherol succinate, beta-tocopherol succinate, gamma-tocopherol succinate, delta-tocopherol succinate, alpha-tocotrienol succinate, beta-tocotrienol succinate, delta-tocotrienol succinate, gamma-tocotrienol succinate, vitamin E TPGS, mixed tocopherols, derivatives, analogs and pharmaceutically acceptable salts thereof.

In a particular aspect, the oil-based composition of the substantially-thickened formulation comprises a combination of a fish oil and vitamin E or an analog of vitamin E.

The therapeutic agent of the substantially-thickened formulation can be one or more of an antioxidant, an anti-inflammatory agent, an anti-coagulant agent, a drug to alter lipid metabolism, an anti-proliferative, an anti-neoplastic, a tissue growth stimulant, a functional protein/factor delivery agent, an anti-infective agent, an imaging agent, an anesthetic agent, a chemotherapeutic agent, a tissue absorption enhancer, an anti-adhesion agent, a germicide, an analgesic, an antiseptic, or pharmaceutically acceptable salts, esters, analogues, derivatives, isomers or pro-drugs thereof. In one embodiment, the therapeutic agent is a Rapamycin derivative, a Rapamycin prodrug, a calcineurin inhibitor, an anti-proliferative, an anti-oxidant, an anti-neoplastic, vitamin E, or an analog of vitamin E, fructose, fish oil, or cetyl alcohol. In another embodiment, the therapeutic agent is Rapamycin, Cyclosporine, Cyclosporine A, a Cyclosporine derivative, or a Rapamycin derivative. The therapeutic agent can also be Rapamycin, Marcaine, Paclitaxel, Cyclosporine, Voclosporine, a Rapamycin prodrug, a Rapamycin derivative and Rifampicin. In another embodiment, the therapeutic agent is in a prodrug form. In one embodiment, the therapeutic agent is ISA 247, TAFA 93 or SAR 943.

In another embodiment the therapeutic agent is a Rapamycin prodrug that is of reduced particle size after fragmentation. In one embodiment, after fragmentation, the Rapamycin prodrug particles have a distribution of size of about 1-15 μm (v,0.1), 16-35 μm (v,0.5), and 36-50 μm (v,0.9).

In another aspect, the invention provides a method for forming a substantially-thickened formulation comprising: (a) associating a composition comprising at least an oil-based composition (e.g., a fish oil) and a therapeutic agent with a cryogenic liquid; (b) fragmenting the composition (e.g., using cryogrinding); (c) returning the composition to an ambient temperature, and (d) shearing the formulation; such that the substantially-thickened formulation is formed. In one embodiment of this method, the step of associating the composition with a cryogenic liquid comprises at least one of suspending, submerging, surrounding, and cooling the composition with the cryogenic liquid. In another embodiment, the cryogenic liquid comprises liquid nitrogen. In yet another embodiment, the composition is fragmented using at least one or more of sonication, grinding, impacting, shearing, shocking, shattering, granulating, pulverizing, shredding, crushing, homogenizing, milling, vibrating, and shaking. In still another embodiment, the method further comprises the step of shearing the composition after the composition is processed.

The therapeutic agent that is used in this method can comprise one or more of an antioxidant, an anti-inflammatory agent, an anti-coagulant agent, a drug to alter lipid metabolism, an anti-proliferative, an anti-neoplastic, a tissue growth stimulant, a functional protein/factor delivery agent, an anti-infective agent, an imaging agent, an anesthetic agent, a chemotherapeutic agent, a tissue absorption enhancer, an anti-adhesion agent, a germicide, an analgesic, an antiseptic, or pharmaceutically acceptable salts, esters, or prodrugs thereof.

In another aspect, the invention provides a method for forming a substantially-thickened formulation comprising: (a) associating a composition comprising a therapeutic agent and a solvent with a cryogenic liquid, wherein the solvent does not dissolve the therapeutic agent; (b) fragmenting the composition; (c) optionally removing the solvent; (d) associating the composition with an oil (e.g., a fish oil); (e) associating the oil-based composition with a cryogenic liquid; (e) fragmenting the oil-based/cryogenic liquid composition (e.g., using cryogrinding); (f) returning the composition to an ambient temperature, and (g) optionally shearing the composition such that a thickened formulation is formed. In one embodiment of this method, the solvent is hexane. In another embodiment of this method, the therapeutic agent is a Rapamycin prodrug. In another embodiment, the composition is sheared such that a thickened formulation is formed.

In one embodiment of the aforementioned process, after fragmentation, the Rapamycin prodrug particles have a distribution of size of about 1-10 μm (v,0.1), 11-30 μm (v,0.5), and 31-50 μm (v,0.9). In another embodiment, after fragmentation, the Rapamycin prodrug particles have a diameter of less than about 10 μm (v,0.9), e.g., a portion of the Rapamycin prodrug particles have a diameter of less than about 10 μm (v,0.9).

In another aspect, the invention provides a medical device, comprising:

a medical device structure; and a coating formed on at least a portion of the medical device structure; wherein the coating comprises a substantially-thickened therapeutic formulation, wherein the substantially-thickened therapeutic formulation comprises an oil-based composition and a therapeutic agent, wherein the therapeutic agent has been reduced by cryogrinding techniques to a solid of a reduced particle size. In one embodiment of this medical device, the therapeutic agent is at a loading concentration that is higher than the maximum solubility concentration of the agent in the oil before cryogrinding. In still another embodiment of the medical device, the therapeutic agent is at a loading concentration of greater than 30% when the solubility of the agent in the oil-based composition before cryogrinding is less than 25%. In yet another embodiment, the therapeutic agent is at a loading concentration of 20%-50%. The medical device can be a vascular graft, hernia mesh, thin film, or stent.

### BRIEF DESCRIPTION OF THE DRAWINGS

The foregoing and other aspects, embodiments, objects, features and advantages of the invention can be more fully understood from the following description in conjunction with the accompanying drawings. In the drawings like reference characters generally refer to like features and structural elements throughout the various figures. The drawings are not necessarily to scale, emphasis instead being placed upon illustrating the principles of the invention.

FIG. 1 is a microscope photo of a substantially-thickened formulation, comprising a Rapamycin pro-drug, fish oil, and vitamin E. As shown in the figure, the therapeutic agent of the substantially-thickened formulation is uniformly dispersed throughout the formulation, such that there is no bulk phase separation between the oil-based composition and the therapeutic agent.

FIGS. 2A and 2B are microscope photos of a substantially-thickened formulation, comprising Rapamycin and cetyl alcohol. As shown in the figures, the therapeutic agent of the substantially-thickened formulation is uniformly dispersed throughout the formulation, such that there is no bulk phase separation between the oil-based composition and the therapeutic agent.

FIGS. 3A and 3B are microscope photos of the substantially-thickened formulation shown in FIGS. 2A and 2B, after washing with a solvent. As shown in the figures, the particle size of the therapeutic agent has been reduced using the cryogrinding process described herein.

### DETAILED DESCRIPTION

In various aspects, embodiments of the present invention include methods for forming a substantially-thickened formulation using a cryogenic liquid. In various embodiments, the method comprises associating a composition comprising at least one dispersing media and a solid therapeutic agent with a cryogenic liquid. The composition is then fragmented. After fragmentation, the composition is returned to room temperature, optionally sheared yielding a substantially-thickened formulation.

Suitable fragmentation methods include, but are not limited to, grinding, shearing, shocking, shattering, granulating, pulverizing, shredding, crushing, homogenizing, sonicating, vibrating, vortexing and/or milling. Suitable means for fragmenting the solid particles include, but are not limited to, mills, e.g., screening mills and impact mills such as hammer mills, homogenizers, e.g., rotor-stator homogenizers, jet mills, colloid mills, high pressure homogenizers and other techniques known to those skilled in the art that impart mechanical energy to dispersions and are capable of resulting in substantial reduction of particle size.

Prior to further describing the invention, it may be helpful to define various terms used herein. In various embodiments, the method of the present invention includes the step of fragmenting compositions while at cryogenic temperatures, thereby forming a substantially-thickened formulation. In various embodiments, a cryogenic fluid is used to bring the compositions to a cryogenic temperature. As used herein the term “cryogenic fluid” or “cryogenic liquid” refers to liquefied gases that are maintained in their liquid state at very low temperatures. Suitable cryogenic liquids of the present invention include, but are not limited to, liquid nitrogen, liquid helium, liquid neon, liquid hydrogen, liquid deuterium, liquid tritium, liquid carbon monoxide, liquid carbon dioxide, liquid fluorine, liquid oxygen, liquid methane, liquid krypton, liquid tetrafluoromethane, liquid ozone, liquid xenon, liquid boron trifluoride, liquid nitrous oxide, liquid ethane, liquid hydrogen chloride, liquid acetylene, liquid fluoroform, liquid 1,1-difluoroethylene, liquid chlortrifluoromethane and liquid argon. In various embodiments, the cryogenic liquid used is liquid nitrogen. In another embodiment, the invention can be performed using a slurry of a solvent (e.g., acetone) and dry ice.

As used herein the term “cryogenic temperature” refers to a temperature which is cold enough to prevent excessive heat generation during processing as well as bringing the solid particles to a temperature where they are embrittled and readily fractured. Typical cryogenic temperatures for these purposes are below about −75° C. It should also be noted that gasses of cryogenic fluids can also be used to maintain a system at a cryogenic temperature. It should be clear to those skilled in the art that suitable temperatures include those that are cold enough to counter heat generation during processing and/or substantially embrittle the material. It should also be obvious to those skilled in the art that other means of cooling, such as dry ice, may be sufficient for certain compositions.

The compositions can be associated with a cryogenic liquid in a variety of ways, including, but not limited to, being suspended, submerged, surrounded, or cooled by a cryogenic liquid. In various embodiments, the composition is directly associated with the cryogenic liquid, i.e., the composition itself is in contact with the cryogenic liquid. The composition can also be indirectly associated with the cryogenic liquid. For example, the composition can be contained in a container, and the container is then suspended, submerged, surrounded, or cooled by a cryogenic liquid.

In various embodiments, the cryogenic liquid is substantially removed after the fragmentation step. One of ordinary skill in the art will recognize methods by which to substantially remove the cryogenic liquid from the composition. For example, the cryogenic liquid, e.g., liquid nitrogen, can be removed by vacuum evaporation or the addition of heat to increase the rate of evaporation. The secondary container can also be removed from the cryogenic environment.

In various embodiments, the solid therapeutic is directly associated with a dispersing media. As used herein, the term “dispersing media” refers to any substance capable of dispersing one or more substances within it. The dispersing media can either be a solvent or a non solvent for the chosen therapeutic agent. Examples of dispersing media include, but are not limited to solvents and non solvents for a chosen substrate, for example, hexane, Isopar, water, ethanol, methanol, NMP, Proglyme, methylene chloride, aceotnitrile, water, acetone, MEK, oils and liquid nitrogen.

The term “nonsolvent” refers to materials (e.g., solvents) that do not dissolve the dispersed substance. The preferred nonsolvents for use in the present invention are hexane and/or oil-based coatings.

Suitable fragmentation methods include, but are not limited to, grinding, shearing, shocking, shattering, granulating, pulverizing, shredding, crushing, homogenizing, sonicating, vibrating, vortexing and/or milling. Suitable means for fragmenting the solid particles include, but are not limited to, mills, e.g., screening mills and impact mills such as hammer mills, and homogenizers, e.g., rotor-stator homogenizers, jet mills, colloid mills, high pressure homogenizers and other techniques known to those skilled in the art that impart mechanical energy to dispersions and are capable of resulting in substantial reduction of particle size. An example of a suitable mill for fragmenting the particles is the Silverson L4R Homogenizer (Silverson Machines, Inc., East Longmeadow, Mass.).

In various embodiments, the solid particles are fragmented by impacting the particles with a rod that is magnetically actuated. For example, a Spex Certiprep Cryomill (model 6750) can be used to fragment solid particles. The composition can be placed in an enclosed vial, and a rod like impactor is enclosed in the vial. The vial is maintained at cryogenic temperatures, and the rod is rapidly oscillated in the vial by means of magnets.

The extent to which particle size is reduced is dependent on the selected processing parameters, the material and the dispersing media. For example, if a Spex Certiprep Cryomill is used to fragment the particles, the size of the vial the composition is contained in, the amount of composition fragmented, and the size of the impactor, the frequency of impact, the number of cycles, the viscosity and glass transition temperature of the materials, the cooling time and the processing temperature will affect the resulting particle size.

In various aspects, a composition comprising a non-polymeric cross-linked gel and at least one dispersing media is associated with a cryogenic liquid and fragmented resulting in a substantially-thickened formulation. The hydrophobic non-polymeric cross-linked gel of the present invention is bio-absorbable. As utilized herein, the term “bio-absorbable” generally refers to having the property or characteristic of being able to penetrate a tissue of a patient's body. In certain embodiments of the present invention, bio-absorption occurs through a lipophilic mechanism. The bio-absorbable substance can be soluble in the phospholipid bi-layer of cells of body tissue.

It should be noted that a bio-absorbable substance is different from a biodegradable substance. Biodegradable is generally defined as capable of being decomposed by biological agents, or capable of being broken down by microorganisms or biological processes. Biodegradable substances can cause inflammatory response due to either the parent substance or those formed during breakdown, and they may or may not be absorbed by tissues.

### Non-Polymeric Cross-Linked Gel

In various aspects, a composition comprising at least one dispersing media and a therapeutic agent is associated with a cryogenic fluid and fragmented, resulting in a substantially-thickened formulation. In various embodiments, the non-polymeric cross-linked gel may be derived from fatty acid compounds. The fatty acids include omega-3 fatty acids wherein the oil utilized to form the gel is fish oil or an analog or derivative thereof. Although some curing methods can have detrimental effects on a therapeutic agent combined with an omega-3 fatty acid oil starting material, one characteristic that can remain after certain curing by, e.g., heating and UV irradiation methods is the non-inflammatory response of tissue when exposed to the cured omega-3 fatty acid material. As such, an oil containing omega-3 fatty acids can be heated, UV irradiated, or both for curing purposes, and still maintain some or even a majority of the therapeutic effectiveness of the omega-3 fatty acids. In addition, although the therapeutic agent combined with the omega-3 fatty acid and cured with the omega-3 fatty acid can be rendered partially ineffective, the portion remaining of the therapeutic agent can, in accordance with the present invention, maintain pharmacological activity and in some cases be more effective than an equivalent quantity of agent delivered with other coating materials.

As liquid fish oil is heated, autoxidation occurs with the absorption of oxygen into the fish oil to create hydroperoxides in an amount dependent upon the amount of unsaturated (C═C) sites in the fish oil. However, the (C═C) bonds are not consumed in the initial reaction. Concurrent with the formation of hydroperoxides is the isomerization of (C═C) double bonds from cis to trans in addition to double bond conjugation. It has been demonstrated that hydroperoxide formation increases with temperature. Heating of the fish oil allows for cross-linking between the fish oil unsaturated chains using a combination of peroxide (C—O—O—C), ether (C—O—C), and hydrocarbon (C—C) bridges. The formation of the cross-links results in gelation of the fish oil. The heating also can also result in the isomerization of cis (C═C) bonds into the trans configuration. The (C═C) bonds can also form C—C cross-linking bridges in the glyceride hydrocarbon chains using a Diels-Alder Reaction. In addition to solidifying the gel through cross-linking, both the hydroperoxide and (C═C) bonds can undergo secondary reactions converting them into lower molecular weight secondary oxidation byproducts including aldehydes, ketones, alcohols, fatty acids, esters, lactones, ethers, and hydrocarbons.

UV initiated curing (photo-oxygenation) in accordance with the present invention involves the interaction between a double bond and singlet oxygen produced from ordinary triplet oxygen by light and typically in the presence of a sensitizer such as chlorophyll or methylene blue and results in the formation of hydroperoxides. The chemical reaction is described in the following graphic.

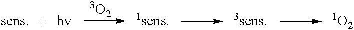

Because the above described reaction is not a radical chain process, it possess no induction period and is typically unaffected by antioxidants commonly used to inhibit autoxidation. However, this reaction can be inhibited by single oxygen quenchers such as carotene. This reaction is limited to C═C carbon atoms and results in a conversion from cis to trans C═C isomers during curing (as occurs with heat initiated curing). However, photo-oxygenation using UV is a relatively quicker reaction than autoxidation from heat curing, in the realm of about 1000-1500 times faster. The quicker reaction especially holds true for methylene interrupted polyunsaturated fatty acids, such as EPA and DHA, which are found in the fish oil-based embodiments of the present invention.

An important aspect of UV curing when compared to heat curing is that although the byproducts obtained by both curing methods are similar, they are not necessarily identical in amount or chemical structure. One reason for this is due to the ability of photo-oxygenation to create hydroperoxides at more possible C═C sites as shown for linolenate in the below graphic.

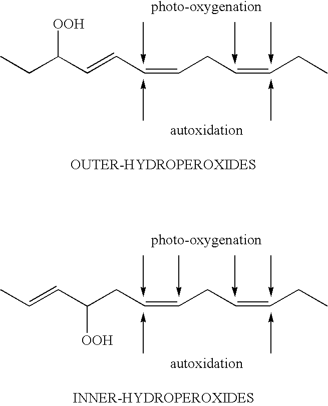

Photo-oxygenation, such as that which results from UV curing, due to its enhanced ability to create inner hydroperoxides, also results in the ability to form relatively greater amounts of cyclic byproducts, which also relates to peroxide cross-linking between fish oil hydrocarbon chains. For example, photo-oxygenation of linolenate results in 6 different types of hydroperoxides to be formed where autoxidation results in only 4. The greater amount of hydroperoxides created using photo-oxygenation results in a similar, but slightly different, structure and amount of secondary byproducts to be formed relative to autoxidation from heat curing. Specifically, these byproducts are aldehydes, ketones, alcohols, fatty acids, esters, lactones, ethers, and hydrocarbons.

Accordingly, in various embodiments, the non-polymeric cross linked gel of the present invention may be derived from fatty acid compounds, such as those of fish oil, that include a cross-linked structure of triglyceride and fatty acid molecules in addition to free and bound glycerol, monoglyceride, diglyceride, and triglyceride, fatty acid, anhydride, lactone, aliphatic peroxide, aldehyde, and ketone molecules. Without being bound by theory, it is believed that there are a substantial amount of ester bonds remaining after curing in addition to peroxide linkages forming the majority of the cross-links in the gel. The non-polymeric cross linked gel degrades (e.g., by hydrolysis) into fatty acid, short and long chain alcohol, and glyceride molecules, which are all substantially non-inflammatory and likewise can be consumable by cells, such as, e.g., smooth muscle cells. Thus, the non-polymeric cross linked gel is bio-absorbable and degrades into substantially non-inflammatory compounds.

The bio-absorbable nature of the gel component of the non-polymeric cross linked gel in the present invention can result in the non-polymeric cross linked gel being absorbed over time by the cells of the body tissue such that substantially none remains. In various embodiments, there are substantially no substances in the non-polymeric cross linked gel, or break down products, that induce an inflammatory response. For example, in various embodiments, the non-polymeric cross linked gel upon break-down does not produce either lactic acid or glycolic acid break-down products in measurable amounts. The preferred non-polymeric cross linked gel is generally composed of, or derived from, omega-3 fatty acids bound to triglycerides, potentially also including a mixture of free fatty acids and vitamin E (alpha-tocopherol). The triglycerides are broken down by lipases (enzymes) which result in free fatty acids that can then be transported across cell membranes. Subsequently, fatty acid metabolism by the cell occurs to metabolize any substances originating with the gel. The bio-absorbable nature of the non-polymeric cross linked gel of the present invention results in the gel being absorbed over time.

An advantage of the cured fish oil in various embodiments of the present invention is that the curing conditions utilized (i.e. cure time and temperature) can directly influence the amount of cross-linking density and byproduct formation, which in turn effects the degradation. Thus, by altering the curing conditions employed, the dissolution rate of a therapeutic compound of interest contained in the non-polymeric cross linked gel can also be altered.

In a various embodiments, an agent, such as, e.g., a free radical scavenger, can be added to the starting material to tailor the drug release profile of the non-polymeric cross linked gel. In various embodiments, vitamin E is added to the starting material to, for example, slow down autoxidation in fish oil by reducing hydroperoxide formation, which can result in a decrease in the amount of cross-linking observed in a cured fish oil gel. In addition, other agents can be used to increase the solubility of a therapeutic agent in the oil component of the starting material, protect the drug from degradation during the curing process, or both. For example vitamin E can also be used to increase the solubility of certain drugs in a fish oil starting material, and thereby facilitate tailoring the drug load of the eventual cured coating. Thus, varying the amount of Vitamin E present in the coating provides an additional mechanism to alter the cross-linking and chemical composition of the non-polymeric cross-linked gel of the present invention.

In various aspects, the non-polymeric hydrophobic cross-linked gel contains the therapeutic agent. In various embodiments, a therapeutic agent is combined with a fatty acid compound prior to formation of a film. The resultant film has the therapeutic agent interspersed throughout.

The hydrophobic non-polymeric cross-linked gel of the present invention is formed from an oil component. The oil component can be either an oil, or an oil composition. The oil components can comprise one or more naturally occurring oils, such as fish oil, cod liver oil, cranberry oil, or other oils having desired characteristics. In various embodiments, the naturally occurring oils, including fish oil, are cured as described herein to form a hydrophobic cross-linked gel stand alone film. In various aspects, the film is suspended in liquid nitrogen and/or an oil-based formulation, and fragmented, thereby reducing the particle size. In various aspects, the film is suspended in liquid nitrogen and/or an oil-based formulation and fragmented, thereby producing a substantially-thickened formulation.

In various embodiments, the present invention makes use of a fish oil in part because of the high content of omega-3 fatty acids, which can provide, e.g., healing support for damaged tissue, as discussed herein. The fish oil can also serve as an anti-adhesion agent. The fish oil can also maintain anti-inflammatory and/or non-inflammatory properties. The present invention is not limited to formation of the hydrophobic non-polymeric cross-linked gel formulation with fish oil as the naturally occurring oil. However, the following description makes reference to the use of fish oil as one example embodiment. Other naturally occurring oils can be utilized in accordance with the present invention.

It should be noted that as utilized herein, the term “fatty acid” includes, but is not limited to, omega-3 fatty acid, fish oil, free fatty acid, monoglycerides, di-glycerides, or triglycerides, esters of fatty acids, or a combination thereof. The fish oil fatty acid includes one or more of arachidic acid, gadoleic acid, arachidonic acid, eicosapentaenoic acid, docosahexaenoic acid or derivatives, analogs and pharmaceutically acceptable salts, esters, or prodrugs thereof.

As utilized herein, the term “free fatty acid” includes, but is not limited to, one or more of butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, methyl and ethyl esters of fatty acids, fatty alcohols, and analogs and pharmaceutically acceptable salts thereof.

With regard to the oils of the present invention, the greater the degree of unsaturation in the fatty acids the lower the melting point of a fat, and the longer the hydrocarbon chain the higher the melting point of the fat. A polyunsaturated fat, thus, has a lower melting point, and a saturated fat has a higher melting point. Those fats having a lower melting point are more often oils at room temperature. Those fats having a higher melting point are more often waxes or solids at room temperature. A fat having the physical state of a liquid at room temperature is an oil. In general, polyunsaturated fats are liquid oils at room temperature, and saturated fats are waxes or solids at room temperature.

Polyunsaturated fats are one of four basic types of lipids extracted by the body from food. The other lipids include saturated fat, as well as monounsaturated fat and cholesterol. Polyunsaturated fats can be further composed of omega-3 fatty acids and omega-6 fatty acids. Unsaturated fatty acids are named according to the position of its first double bond of carbons, those fatty acids having their first double bond at the third carbon atom from the methyl end of the molecule are referred to as omega-3 fatty acids. Likewise, a first double bond at the sixth carbon atom is called an omega-6 fatty acid. There can be both monounsaturated and polyunsaturated omega fatty acids.

Omega-3 and omega-6 fatty acids are also known as essential fatty acids because they are important for maintaining good health, despite the fact that the human body cannot make them on its own. As such, omega-3 and omega-6 fatty acids must be obtained from external sources, such as food. Omega-3 fatty acids can be further characterized as containing eicosapentaenoic acid (EPA), docosahexanoic acid (DHA), and alpha-linolenic acid (ALA). EPA and DHA both have anti-inflammatory effects and wound healing effects within the human body.

### Substantially-Thickened Formulations

In various aspects, the method of the present invention includes the step of fragmenting compositions while associated with a cryogenic fluid, e.g., liquid nitrogen, thereby forming a substantially-thickened formulation. In various embodiments, the compositions fragmented include, but are not limited to, therapeutic agents dispersed in at least one dispersing media. For example, compositions fragmented may include, a dispersing media, e.g., a mixture of fish oil and vitamin E, and at least one of vitamin E, d-alpha-tocopheryl polyethylene glycol succinate (vitamin E TPGS), Rapamycin, prodrugs of Rapamycin, and fructose dispersed therein. In various embodiments, compositions fragmented may include fish oil, linseed oil, lecithin, fish gelatin and fatty alcohols.

As used herein, the term “substantially-thickened formulation” refers to a composition, e.g., a therapeutic agent dispersed in at least dispersing media, which exhibits an increased viscosity after being associated with a cryogenic liquid, e.g., liquid nitrogen, and fragmented. In various embodiments, substantially-thickened formulations of the present invention exhibit shear thinning, i.e., a decrease in viscosity with an increasing rate of shear. The rheology of the substantially-thickened formulation is dependent on a plurality of factors including, but not limited to, the type of therapeutic agent, the concentration of the therapeutic agent, the particle size of the therapeutic agent, the dispersion media used, the solubility of the dispersed particle in the dispersion media, the material properties and the processing conditions. The material properties include, but are not limited to, the initial particle size, crystallinity, surface chemistry, and surface charge.

The rheology of dispersions is dependent upon a number of factors including, but not limited to, the properties of the dispersing fluid and the dispersed phase. The stability of a dispersion, that is, its resistance to separation and/or flocculation, is connected to the rheology of the system because they both depend upon the interactive forces between particles. An attraction between particles may cause the particles to aggregate or settle whereas repulsive forces may prevent these events. The strength of attraction can be modulated by changes that include, but are not limited to, pH, surface charge, particle shape, particle size, and intrinsic properties of the dispersing phase. In the substantially-thickened formulations of this application, the material behaves as if it were a solid below a certain critical level of stress. Above this critical level (known as its yield stress) its properties change abruptly. Above this yield stress, the material behaves as a fluid.

Cryogrinding alters the interactive forces between particles in dispersions. For example, a Rapamycin drug dispersion that was not cryoground was tested on an Anton-Paar MCR 301 series rheometer. A frequency sweep was conducted and it was found that the material's rheology was consistent with a thixotropic fluid at all strains. A Rapamycin pro-drug dispersion at similar concentrations was cryoground using a Spex Certiprep Cryomill and was tested on the same Anton-Paar MCR series rheometer. Its frequency sweep exhibited a sharp transition from the rheology of a solid to a fluid. This is consistent with altered interactive forces between the particles that led to the creation of a substantially-thickened formulation that behaves much like a solid below a critical yield stress. The equivalent unprocessed formulation behaved as a fluid.

Simple particle size changes for these formulations do not result in a substantially-thickened formulation. Rapamycin pro-drug was air-jet milled to achieve sub-micron particles. These particles were dispersed in fish oil:Vitamin E at a 27% concentration but not cryoground. The rheology of this dispersion was consistent with that of a fluid. Cryogrinding of this fluid led to the creation of a substantially-thickened formulation with a defined yield stress. Rapamycin pro-drug cryoground in fish oil:Vitamin E formulations achieve sub-micron particle sizes as well and do exhibit the behavior of substantially-thickened formulations (that is, solid-like below a critical yield stress). Thus, particle size reduction alone does not impart the rheological behavior of substantially-thickened formulations. Cryogrinding is necessary to alter the inter-particle interactions so that the rheology is suitably altered.

In a substantially-thickened formulation that is processed with the method of the current invention there is a high viscosity with suspended particles. If sufficient heat is applied to the formulation the interparticle interactions will break down and the particles will no longer be suspended in the oil-based matrix. Upon secondary cryogenic processing the suspended particles and thickened viscosity can be regained. The substantially-thickened formulation can also be described as having an oil phase that is suspended in a structuring network. The association of the particles in the formulation has the ability to create a structure that can entrap the oil phase of the formulation. This association of particles and entrapment of the oil phase can lead to the creation of a substantially-thickened formulation.

Cryogenic processing can be used to suspend particles in an oil-based matrix at various concentrations. As the particulate loading increases the viscosity of the formulation will also increase. At a given particle loading the material will become dry and will be more useful for compression formed applications. When particulates are processed in the absence of a dispersing fluid, such as an oil-based matrix, the particles will have a tendency to flatten and not form a substantially-thickened formulation.

Cryogrinding in an oil medium can reduce or eliminate the need for strict environmental control over humidity when processing materials which are prone to hydrolysis. To demonstrate this, Rapamycin pro-drug was cryoground at 60% relative humidity and showed a shift in the OH FTIR band indicating possible hydrolysis of the drug. Under low humidity (6%) this hydrolysis did not occur. When the Rapamycin pro-drug was ground at 60% relative humidity in a 70:30 Fish Oil:vitamin E formulation, hydrolysis did not occur.

### Compositions

In various aspects, compositions comprising a therapeutic agent dispersed in at least one dispersing media, are suspended in liquid nitrogen and fragmented, thereby resulting in a substantially-thickened formulation. In various embodiments, the dispersing media is an oil. The oil or oils used may include any oil, for example, naturally occurring oils, such as fish oil, cod liver oil, cranberry oil, linseed oil, lecithin, or other oils having desired characteristics. In various embodiments, the oil may include, but is not limited to, fish oil, vitamin E, and/or their derivatives, and combinations thereof. In various embodiments, the therapeutic agent is dispersed in a mixture of fish oil and vitamin E. The two components of a two base component oil mixture may be in any ratio, including, but not limited to, 0:100, 10:90, 20:80, 30:70, 40:60, 50:50, 60:40, 70:30, 80:20, 90:10, 100:0 fish oil to vitamin E. Additional components, including, but not limited to, therapeutic agents, can be added to such two base component oil mixtures. It will also be obvious to those skilled in the art that systems containing more than two base components can be used.

In various embodiments, the ratio of fish oil to vitamin E affects the recovery of initial viscosity of the substantially-thickened formulation after it is exposed to shear. For example, substantially-thickened formulations with a higher vitamin E concentration recover their initial viscosity after shearing more than formulations with a lower vitamin E concentration with selected therapeutic agents.

In various embodiments, the ratio of fish oil to vitamin E in the composition impacts the effect processing conditions have on the viscosity of the substantially-thickened formulation. For example, substantially-thickened formulations made with certain particles and a higher vitamin E concentration exhibit a decrease of initial viscosity with an increase in temperature whereas formulations with lower vitamin E have a viscosity that is substantially independent of temperature over a range between about 18° C. to about 50° C.

In various embodiments, the concentration of the therapeutic agent dispersed in the oil affects the viscosity of the substantially-thickened formulation. For example, the higher the concentration of the therapeutic agent dispersed in the oil or oil mixture, the more viscous the formulation. In various embodiments, a maximum concentration is reached for a given dispersed ingredient before it can no longer be sheared into a substantially-thickened formulation.

In various embodiments, cryogrinding in an oil medium can reduce or eliminate the need for strict environmental control over humidity when processing materials that are prone to hydrolysis. To demonstrate this, Rapamycin pro-drug was cryoground at 60% relative humidity and showed a shift in the OH band of the FTIR spectra indicating possible hydrolysis of the drug. Under low humidity (6%) this hydrolysis did not occur. When the Rapamycin pro-drug was ground at 60% relative humidity in a 70:30 Fish Oil:vitamin E formulation, hydrolysis did not occur.

### Cyrogenic Fragmentation

In various aspects, a composition comprising a therapeutic agent and a dispersing media is associated with a cryogenic fluid and fragmented. The dispersing media is selected based on the solid therapeutic agent used. For example, the dispersing media selected is one that does not dissolve the therapeutic agent, and is easily evaporated. In various embodiments the dispersing media is hexane. In various embodiments, the dispersing media is liquid nitrogen. (It should be noted that liquid nitrogen should never be sealed in a non vented container.)

In various aspects, the method of the present invention includes the step of fragmenting compositions while associated with a cryogenic fluid, e.g., liquid nitrogen, thereby resulting in a substantially-thickened formulation. In various embodiments, the compositions include, but are not limited to, therapeutic agents combined with a dispersing media.

Suitable fragmentation methods include, but are not limited to, grinding, shearing, shocking, shattering, granulating, pulverizing, shredding, crushing, homogenizing, sonicating, vibrating, vortexing and/or milling. Suitable means for fragmenting the solid particles include, but are not limited to, mills, e.g., screening mills and impact mills such as hammer mills, and homogenizers, e.g., rotor-stator homogenizers, jet mills, colloid mills, high pressure homogenizers and other techniques known to those skilled in the art that impart mechanical energy to dispersions and are capable of resulting in substantial reduction of particle size. The compositions may be fragmented for a number of cycles, i.e., fragmenting the compositions for a specified period of time, followed by a specified period of time in which the compositions are allowed to cool back to cryogenic temperatures. In various embodiments, the time period for the system to cool back to cryogenic temperatures is about 1 to about 2 minutes.

For example the compositions may be fragmented for 1, 2, 3, 4, 5, 6, 7, 8, 9, or 10 cycles. In various embodiments, the compositions are fragmented from 4 to 8 cycles. In various embodiments, an increase in fragmentation cycles results in an increase in the viscosity of the substantially-thickened formulations. In various embodiments, the fragmentation cycles are from about 1 to about 2 minutes per cycle.

In various embodiments, the increase in viscosity of the substantially-thickened formulation may be partially reversed by application of heat. For example, when the temperature of a substantially-thickened formulation is increased, the viscosity of the substantially-thickened formulation may decrease. Upon cooling, the substantially-thickened formulation does not regain an increased viscosity. The heating of the substantially-thickened formulation results in an irreversible loss of viscosity that can be regained with repeated cryo processing.

In various embodiments, the particle size of the therapeutic agent used is one of a plurality of factors that affects the viscosity of the substantially-thickened formulation.

In various aspects, a composition comprising a therapeutic agent and a dispersing media are associated with a cryogenic fluid and fragmented, resulting in a substantially-thickened formulation. The therapeutic agent can be a non-polymeric cross linked gel as described herein. The therapeutic agent can also be a non-polymeric cross linked and an additional therapeutic agent.

In one aspect, the invention provides a method of preparing a substantially-thickened therapeutic formulation comprising an oil-based composition and a therapeutic agent, wherein the therapeutic agent is of a reduced particle size. The method comprises loading a therapeutic agent, such as a Rapamycin pro-drug or calcineurin inhibitor, into a dispersing media that comprises fish oil. The fish oil can optionally include vitamin E. The resulting formulation is cryo-processed using, for example, a cryomill. The cryo-processed material is warmed to room temperature, and sheared. In one embodiment, the therapeutic agent can be mixed with a solvent, such as acetone, before mixing with the dispersing media.

In another embodiment of the method, the fish oil, with or without a therapeutic agent, is cured to a film using heat or UV radiation. This film is then added to a dispersing media, such as fish oil, cryo-processed using, for example, a cryomill, warmed to room temperature, and optionally sheared, resulting in a substantially-thickened therapeutic formulation comprising an oil-based composition and a therapeutic agent, wherein the therapeutic agent is of a reduced particle size.

As utilized herein, the phrase “therapeutic agent(s)” refers to a number of different drugs or agents available, as well as future agents that may be useful in the present invention. In various embodiments, the therapeutic agent comprises saturated fatty acids, unsaturated fatty acids, fatty alcohols, esters of fatty acids, non-polymeric cross linked gels, polymers, vitamin E acetate, vitamin E succinate, mixed tocopherols, lecithins, PLA and gelatin.

The therapeutic agent component can take a number of different forms including antioxidants, anti-inflammatory agents, anti-coagulant agents, drugs to alter lipid metabolism, anti-proliferatives, anti-neoplastics, tissue growth stimulants, functional protein/factor delivery agents, anti-infective agents, imaging agents, anesthetic agents, chemotherapeutic agents, tissue absorption enhancers, anti-adhesion agents, germicides, antiseptics, proteoglycans, GAG's, gene delivery (polynucleotides), antifibrotics, analgesics, prodrugs, polysaccharides (e.g., heparin), anti-migratory agents, pro-healing agents, and ECM/protein production inhibitors. The therapeutic agent component can alternatively take the form of an agent selected from the group consisting of cerivastatin, cilostazol, fluvastatin, lovastatin, Paclitaxel, pravastatin, Rapamycin, and simvastatin, and any additional desired therapeutic agents such as those listed in Table 1 below.

TABLE 1

| CLASS | EXAMPLES |
|---|---|
| Antioxidants | Alpha-tocopherol, lazaroid, probucol, phenolic antioxidant, |
| | resveretrol, AGI-1067, vitamin E, mixed tocopherols |
| Antihypertensive Agents | Diltiazem, nifedipine, verapamil |
| Antiinflammatory Agents | Glucocorticoids (e.g. dexamethazone, |
| | methylprednisolone), leflunomide, NSAIDS, ibuprofen, |
| | acetaminophen, hydrocortizone acetate, hydrocortizone |
| | sodium phosphate, macrophage-targeted bisphosphonates, |
| | Cyclosporine, Voclosporine |
| Growth Factor | Angiopeptin, trapidil, suramin |
| Antagonists | |
| Antiplatelet Agents | Aspirin, dipyridamole, ticlopidine, clopidogrel, GP IIb/IIIa |
| | inhibitors, abcximab |
| Anticoagulant Agents | Bivalirudin, heparin (low molecular weight and |
| | unfractionated), wafarin, hirudin, enoxaparin, citrate |
| Thrombolytic Agents | Alteplase, reteplase, streptase, urokinase, TPA, citrate |
| Drugs to Alter Lipid | Fluvastatin, colestipol, lovastatin, atorvastatin, amlopidine |
| Metabolism (e.g. statins) | |
| ACE Inhibitors | Elanapril, fosinopril, cilazapril |
| Antihypertensive Agents | Prazosin, doxazosin |
| Antiproliferatives and | Cyclosporine, cochicine, mitomycin C, sirolimus |
| Antineoplastics | micophenonolic acid, Rapamycin, everolimus, tacrolimus, |
| | Paclitaxel, QP-2, actinomycin, estradiols, dexamethasone, |
| | methatrexate, cilostazol, prednisone, Cyclosporine, |
| | doxorubicin, ranpirnas, troglitzon, valsarten, pemirolast, C- |
| | MYC antisense, angiopeptin, vincristine, PCNA ribozyme, |
| | 2-chloro-deoxyadenosine, Voclosporine |
| Tissue growth stimulants | Bone morphogeneic protein, fibroblast growth factor |
| Promotion of hollow | Alcohol, surgical sealant polymers, polyvinyl particles, 2- |
| organ occlusion or | octyl cyanoacrylate, hydrogels, collagen, liposomes |
| thrombosis | |
| Functional Protein/Factor | Insulin, human growth hormone, estradiols, nitric oxide, |
| delivery | endothelial progenitor cell antibodies |
| Second messenger | Protein kinase inhibitors |
| targeting | |
| Angiogenic | Angiopoetin, VEGF |
| Anti-Angiogenic | Endostatin |
| Inhibition of Protein | Halofuginone, prolyl hydroxylase inhibitors, C-proteinase |
| Synthesis/ECM formation | inhibitors |
| Antiinfective Agents | Penicillin, gentamycin, adriamycin, cefazolin, amikacin, |
| | ceftazidime, tobramycin, levofloxacin, silver, copper, |
| | hydroxyapatite, vancomycin, ciprofloxacin, rifampin, |
| | mupirocin, RIP, kanamycin, brominated furonone, algae |
| | byproducts, bacitracin, oxacillin, nafcillin, floxacillin, |
| | clindamycin, cephradin, neomycin, methicillin, |
| | oxytetracycline hydrochloride, Selenium. |
| Gene Delivery | Genes for nitric oxide synthase, human growth hormone, |
| | antisense oligonucleotides |
| Local Tissue perfusion | Alcohol, H2O, saline, fish oils, vegetable oils, liposomes |
| Nitric oxide Donor | NCX 4016 - nitric oxide donor derivative of aspirin, |
| Derivatives | SNAP |
| Gases | Nitric oxide, compound solutions |
| Imaging Agents | Halogenated xanthenes, diatrizoate meglumine, diatrizoate |
| | sodium |
| Anesthetic Agents | Lidocaine, benzocaine |
| Descaling Agents | Nitric acid, acetic acid, hypochlorite |
| Anti-Fibrotic Agents | Interferon gamma-1b, Interluekin-10 |
| Immunosuppressive/Immunomodulatory | Cyclosporine, Rapamycin, mycophenolate motefil, |
| Agents | leflunomide, tacrolimus, tranilast, interferon gamma-1b, |
| | mizoribine, Voclosporine |
| Chemotherapeutic Agents | Doxorubicin, Paclitaxel, tacrolimus, sirolimus, fludarabine, |
| | ranpirnase |
| Tissue Absorption | Fish oil, squid oil, omega 3 fatty acids, vegetable oils, |
| Enhancers | lipophilic and hydrophilic solutions suitable for enhancing |
| | medication tissue absorption, distribution and permeation |
| Anti-Adhesion Agents | Hyaluronic acid, human plasma derived surgical |
| | sealants, and agents comprised of hyaluronate and |
| | carboxymethylcellulose that are combined with |
| | dimethylaminopropyl, ehtylcarbodimide, hydrochloride, |
| | PLA, PLGA |
| Ribonucleases | Ranpirnase |
| Germicides | Betadine, iodine, sliver nitrate, furan derivatives, |
| | nitrofurazone, benzalkonium chloride, benzoic acid, |
| | salicylic acid, hypochlorites, peroxides, thiosulfates, |
| | salicylanilide |
| Antiseptics | Selenium |
| Analgesics | Bupivicaine, naproxen, ibuprofen, acetylsalicylic acid |

The therapeutic agent can be an active agent as contained in the non-polymeric crosslinked gel. Pharmaceutically acceptable salts, esters, isomers or prodrugs of the therapeutic agent are also suitable for use in the present invention. In various embodiments, the non-polymeric cross linked gel itself comprises the therapeutic agent.

In various embodiments, the therapeutic agent comprises an mTOR targeting compound. The term “mTOR targeting compound” refers to any compound which modulates mTOR directly or indirectly. An example of an “mTOR targeting compound” is a compound that binds to FKBP 12 to form, e.g., a complex, which in turn inhibits phosphoinostide (PI)-3 kinase, that is, mTOR. In various embodiments, mTOR targeting compounds inhibit mTOR. Suitable mTOR targeting compounds include, for example, Rapamycin and its derivatives, analogs, prodrugs, esters and pharmaceutically acceptable salts thereof.

Calcineurin is a serine/threonine phospho-protein phosphatase and is composed of a catalytic (calcineurin A) and regulatory (calcineurin B) subunit (about 60 and about 18 kDa, respectively). In mammals, three distinct genes (A-alpha, A-beta, A-gamma) for the catalytic subunit have been characterized, each of which can undergo alternative splicing to yield additional variants. Although mRNA for all three genes appears to be expressed in most tissues, two isoforms (A-alpha and A-beta) are most predominant in brain.

The calcineurin signaling pathway is involved in immune response as well as apoptosis induction by glutamate excitotoxicity in neuronal cells. Low enzymatic levels of calcineurin have been associated with Alzheimer's disease. In the heart or in the brain calcineurin also plays a key role in the stress response after hypoxia or ischemia.

Substances which are able to block the calcineurin signal pathway can be suitable therapeutic agents for the present invention. Examples of such therapeutic agents include, but are not limited to, FK506, tacrolimus, Cyclosporine, Voclosporine and include derivatives, analogs, esters, prodrugs, pharmaceutically acceptably salts thereof, and conjugates thereof which have or whose metabolic products have the same mechanism of action. Further examples of cyclosporin include, but are not limited to, naturally occurring and non-natural cyclosporins prepared by total- or semi-synthetic means or by the application of modified culture techniques. The class comprising cyclosporins includes, for example, the naturally occurring Cyclosporins A through Z, as well as various non-natural cyclosporin derivatives, artificial or synthetic cyclosporin derivatives. Artificial or synthetic cyclosporins can include dihydrocyclosporins, derivatized cyclosporins, and cyclosporins in which variant amino acids are incorporated at specific positions within the peptide sequence, for example, dihydro-cyclosporin D.

In various embodiments, the therapeutic agent comprises one or more of a mTOR targeting compound and a calcineurin inhibitor. In various embodiments, the mTOR targeting compound is a Rapamycin or a derivative, analog, ester, prodrug, pharmaceutically acceptably salts thereof, or conjugate thereof which has or whose metabolic products have the same mechanism of action. In various embodiments, the calcineurin inhibitor is a compound of Tacrolimus, or a derivative, analog, ester, prodrug, pharmaceutically acceptably salts thereof, or conjugate thereof which has or whose metabolic products have the same mechanism of action or a compound of Cyclosporin or a derivative, analog, ester, prodrug, pharmaceutically acceptably salts thereof, or conjugate thereof which has or whose metabolic products have the same mechanism of action.

In various embodiments, the therapeutic agent comprises an anti-adhesive agent. As used herein, the term “anti-adhesion agent” refers to any compound that prevents adhesions or accretions of body tissues formed in response to injury of various kinds, e.g., surgery, infection, chemotherapy, radiation. Anti-adhesion agents of the present invention include, but are not limited to, hyaluronic acid, human plasma derived surgical sealants, and agents comprised of hyaluronate and carboxymethylcellulose that are combined with dimethylaminopropyl, ehtylcarbodimide, hydrochloride, PLA, and/or PLGA.

In various embodiments, the therapeutic agent comprises an antiproliferative and/or an antineoplastic agent. The term “antiproliferative/antineoplastic agent” as used herein refers to any compound which inhibits or prevents the growth or development of cells, e.g., smooth muscle cells, or malignant cells. Suitable antiproliferative and antineoplastic agents include, but are not limited to, Paclitaxel or its derivatives, analogs, esters, prodrugs, and pharmaceutically acceptable salts thereof.

A therapeutically effective amount refers to that amount of a compound sufficient to result in amelioration of symptoms, e.g., treatment, healing, prevention or amelioration of the relevant medical condition, or an increase in rate of treatment, healing, prevention or amelioration of such conditions. When applied to an individual active ingredient, administered alone, a therapeutically effective amount refers to that ingredient alone. When applied to a combination, a therapeutically effective amount can refer to combined amounts of the active ingredients that result in the therapeutic effect, whether administered in combination, serially or simultaneously. In various embodiments, where formulations comprise two or more therapeutic agents, such formulations can be described as a therapeutically effective amount of compound A for indication A and a therapeutically effective amount of compound B for indication B, such descriptions refer to amounts of A that have a therapeutic effect for indication A, but not necessarily indication B, and amounts of B that have a therapeutic effect for indication B, but not necessarily indication A.

Actual dosage levels of the active ingredients in the compositions of the present invention may be varied so as to obtain an amount of the active ingredients which is effective to achieve the desired therapeutic response without being unacceptably toxic. The selected dosage level will depend upon a variety of pharmacokinetic factors including the activity of the particular therapeutic agent (drug) employed, or the ester, salt or amide thereof, the mechanism of drug action, the time of administration, the drug release profile of the coating, the rate of excretion of the particular compounds being employed, the duration of the treatment, other drugs, compounds and/or materials used in combination with the particular compounds employed, and like factors known in the medical arts.

As used herein, the term “solid therapeutic agent” refers to therapeutic agents in solid form, i.e., not liquids or gases.

In one embodiment, the therapeutic agent is stabilized. The biologically active agent can be stabilized against degradation, loss of potency and/or loss of biological activity, all of which can occur during formation of the microparticles having the biologically active agent dispersed therein, and/or prior to and during in vivo release of the biologically active agent from the microparticles. In one embodiment, stabilization can result in a decrease in the solubility of the biologically active agent, the consequence of which is a reduction in the initial release of the biologically active agent, in particular, when release is from microparticles for sustained release of the biologically active agent. In addition, the period of release of the biologically active agent from the microparticles can be prolonged.

Stabilization of the biologically active agent can be accomplished, for example, by the use of a stabilizing agent or a specific combination of stabilizing agents. “Stabilizing agent,” as that term is used herein, is any agent which binds or interacts in a covalent or non-covalent manner or is included with the biologically active agent.

In another embodiment, the stabilizing agent can be vitamin E. It should be noted that as utilized herein to describe the present invention, the term “vitamin E” and the term “alpha-tocopherol,” are intended to refer to the same or substantially similar substance, such that they are interchangeable and the use of one includes an implicit reference to both. Further included in association with the term vitamin E are such variations including but not limited to one or more of alpha-tocopherol, beta-tocopherol, delta-tocopherol, gamma-tocopherol, alpha-tocotrienol, beta-tocotrienol, delta-tocotrienol, gamma-tocotrienol, alpha-tocopherol acetate, beta-tocopherol acetate, gamma-tocopherol acetate, delta-tocopherol acetate, alpha-tocotrienol acetate, beta-tocotrienol acetate, delta-tocotrienol acetate, gamma-tocotrienol acetate, alpha-tocopherol succinate, beta-tocopherol succinate, gamma-tocopherol succinate, delta-tocopherol succinate, alpha-tocotrienol succinate, beta-tocotrienol succinate, delta-tocotrienol succinate, gamma-tocotrienol succinate, mixed tocopherols, vitamin E TPGS, derivatives, analogs and pharmaceutically acceptable salts thereof.

“Sustained release,” as that term is used herein, is a release of the biologically active agent from the microparticles which occurs over a period which is longer than the period during which a biologically significant amount of the active agent would be available following direct administration of the active agent, e.g., a solution or suspension of the active agent. In various embodiments, a sustained release is a release of the biologically active agent which occurs over a period of at least about one day such as, for example, at least about 2, 4, 6, 8, 10, 15, 20, 30, 60, or at least about 90 days. A sustained release of the active agent can be a continuous or a discontinuous release, with relatively constant or varying rates of release. The continuity of release and level of release can be affected by the biologically active agent loading, and/or selection of excipients to produce the desired effect.

“Sustained release,” as used herein, also encompasses “sustained action” or “sustained effect.” “Sustained action” and “sustained effect,” as those terms are used herein, refer to an increase in the time period over which the biologically active agent performs its therapeutic, prophylactic and/or diagnostic activity as compared to an appropriate control. “Sustained action” is also known to those experienced in the art as “prolonged action” or “extended action.”

### EXEMPLIFICATION OF THE INVENTION

The invention is further illustrated by the following examples. The examples should not be construed as further limiting.

### Example 1

A Rapamycin pro-drug (TAFA 93) was loaded at 40% into a dispersing media that consisted of 70% fish oil and 30% vitamin E. 1.5 grams of this formulation were placed into a vial and cryo-processed using a Spex Certiprep cryomill. The sample was processed for 4 cycles with cooling times of two minutes between cycles and a frequency of 20 impacts per second. The resulting material was warmed to room temperature, sheared and removed from the vial. The resulting formulation was highly viscous and shear thinning with small crystals was evident throughout the sample. A similar formulation containing a 70% Fish oil 30% Vitamin E dispersing media was formulated at 23% pro-drug using a soluble technique. For this technique, 230 mg of the pro-drug was dissolved in ig of acetone and mixed with 770 mg of the dispersing media. After removal of the solvent the formulation was found to crack and phase separate due to the limited solubility of the pro-drug in the dispersing media. This example demonstrates that a higher uniform drug loading is attainable using a cryogrinding technique than is possible with a soluble technique when this pro-drug and this base coating are used.

### Example 2

A Rapamycin derivative (SAR 943) was loaded at 31% into a dispersing media that consisted of 70% fish oil and 30% vitamin E. 1.5 grams of this formulation were placed into a vial and cryo-processed using a Spex Certiprep cryomill. The sample was processed for 8 cycles with cooling times of two minutes between cycles and a frequency of 20 impacts per second. The resulting material was warmed to room temperature, sheared and removed from the vial. The resulting formulation was highly viscous and shear thinning with small crystals evident throughout the sample.

### Example 3

A calcineurin inhibitor was loaded at 40% into a dispersing media that consisted of 70% fish oil and 30% vitamin E. 1.5 grams of this formulation were placed into a vial and cryo-processed using a Spex Certiprep cryomill. The sample was processed for 8 cycles with cooling times of two minutes between cycles and a frequency of 20 impacts per second. The resulting material was warmed to room temperature, sheared and removed from the vial. The resulting formulation was highly viscous with a Newtonian flow behavior.

### Example 4

Vitamin E TPGS was loaded at 30% into a dispersing media that consisted of 100% fish oil. 1.5 grams of this formulation were placed into a vial and cryo-processed using a Spex Certiprep cryomill. The sample was processed for 4 cycles with cooling times of two minutes between cycles and a frequency of 20 impacts per second. The resulting material was warmed to room temperature, sheared and removed from the vial. The resulting formulation was highly viscous upon standing and was extremely shear thinning.

### Example 5

Fructose was loaded at 40% into a dispersing media that consisted of 100% fish oil. 1.5 grams of this formulation were placed into a vial and cryo-processed using a Spex Certiprep cryomill. The sample was processed for 8 cycles with cooling times of two minutes between cycles and a frequency of 20 impacts per second. The resulting material was warmed to room temperature, sheared and removed from the vial. The resulting formulation was highly viscous upon standing and was extremely shear thinning.

### Example 6

Fish oil was cured to a film with the use of heat. It was then loaded at 35% into a dispersing media that consisted of 100% fish oil. 1.5 grams of this formulation was placed into a vial and cryo-processed using a Spex Certiprep cryomill. The sample was processed for 8 cycles with cooling times of two minutes between cycles and a frequency of 30 impacts per second. The resulting material was warmed to room temperature, sheared and removed from the vial. The resulting formulation was highly viscous and sticky.

### Example 7

Cetyl Alcohol was loaded at 30% into a dispersing media that consisted of 70% fish oil and 30% Vitamin E. 1.5 grams of this formulation were placed into a vial and cryo-processed using a Spex Certiprep cryomill. The sample was processed for 8 cycles with cooling times of two minutes between cycles and a frequency of 30 impacts per second. The resulting material was warmed to room temperature, sheared and removed from the vial. The resulting formulation was highly viscous and smooth.

### Example 8

A Rapamycin Pro-drug and a Rapamycin Derivative were evaluated before and after cryo-processing to determine some of the factors that are contributing to the substantial thickening of the formulations. The therapeutic agents were analyzed for particle size prior to cryogrinding to establish a base line. Both were dispersed in 70:30 Fish oil:vitamin E and processed via cryogrinding. The particle size analyses follow. (The Rapamycin prodrug is TAFA 93, and the Rapamycin derivative is SAR 943.)

TABLE B
PARTICLE SIZE REDUCTION DATA

| | | | | Volume Weighted | | |
|---|---|---|---|---|---|---|
| | Drug | | Analysis | Diameters (um) | | |
| Sample Description | form | | Equipment | D(v, 0.1) | D(v, 0.5) | D(v, .9) |
| Rapamycin prodrug powder | Dry | | Pharmavision | 121.62 | 529.34 | 1613.84 |
| Rapamycin prodrug | In | 70% | Accusizer 770 | 11.01 | 28.38 | 41.91 |
| cryoground (40%) | FO | 30% | | | | |
| | Vit E | | | | | |
| Rapamycin derivative | Dry | | Accusizer 770 | 4.51 | 7.88 | 27.32 |
| powder | | | | | | |
| Rapamycin derivative | In | 70% | Microtrac | 0.61 | 1.55 | 2.399 |
| cryoground | FO | 30% | | | | |

Vit E

The samples that were cryo processed resulted in substantially-thickened formulations and had smaller and more uniform particle size of the dispersed phase therapeutic agent.

### Example 9

532 mg of a Rapamycin pro-drug was suspended in 692 mg of hexane. The mixture was subjected to cryogrinding for 8 cycles, each lasting 2.0 minutes and with a 2.0 minute between-cycle cool down period in liquid nitrogen in order to maintain the mixture at cryogenic temperatures. The impactor speed was set at 15 for a frequency of impact of 30 cycles per second. After the cryogrinding was completed, the sample was brought to room temperature and the hexane was allowed to evaporate. The particles were analyzed for size using appropriate instrumentation. The initial sample was analyzed using a vision system (Pharmavision) whereas the processed sample was able to be analyzed using light obscuration techniques due to its smaller size (Accusizer 770).

| | D(v, 0.1) | D(v, 0.5) | D(v, .9) | |
|---|---|---|---|---|
| Before | 151.53 | 361.09 | 1016.38 | Pharmavision |
| cryogrinding | | | | |
| After | 5.96 | 20.31 | 39.64 | Accusizer |
| cryogrinding | | | | |

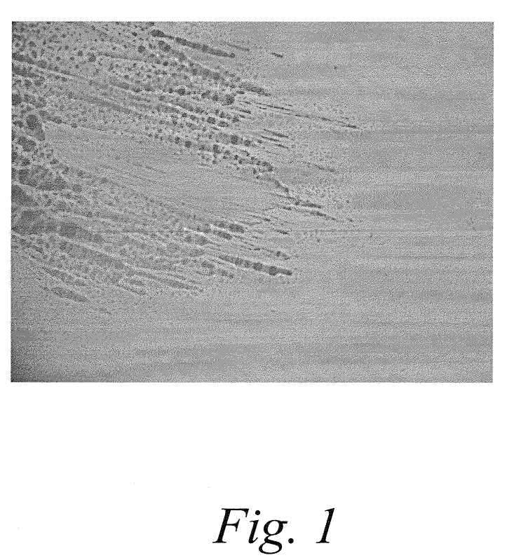

Therapeutic agent that was prepared in this manner was then loaded at 34.1% as a dispersed phase in a continuous phase of 70% fish oil 30% vitamin E. The formulation could then be cryo-processed for fewer cycles to obtain a uniform substantially-thickened formulation. As shown in FIG. 1, the mixture was very homogeneous.

### Example 10

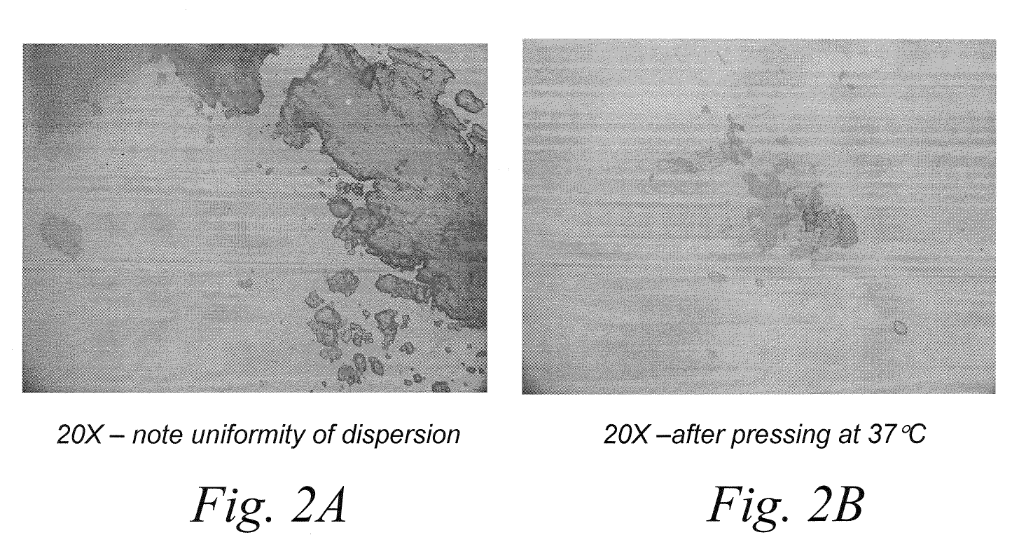

316 mg of Rapamycin was placed in a cryogrinding vial with 753 mg of cetyl alcohol. The mixture was heterogeneous. It was processed in a Spex Certiprep cryogrinder for 8 cycles, each cycle lasting 2.0 minutes and with a 2.0 minute between-cycle cool down to maintain the system at cryogenic temperatures. After bring to room temperature, the mixture was found to be completely homogenous, that is, it was a solid homogeneously dispersed within a second solid. With gentle heat and pressure, it could be smeared into a thin film. Thus, the continuous phase was the cetyl alcohol. (See FIGS. 2A and 2B.)

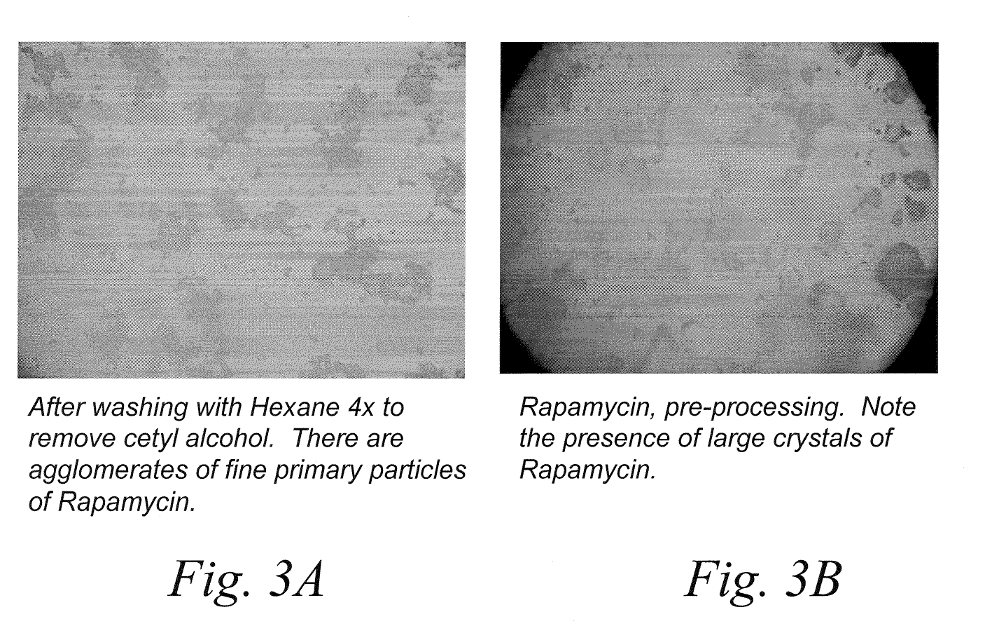

The Rapamycin material that was cryoground with the cetyl alcohol was then washed with 4 exchanges of hexane allowing all of the cetyl alcohol to dissolve. When the washed Rapamycin was placed on a microscope slide it was determined that the particle size was substantially smaller than non processed material. (See FIGS. 3A and 3B.)

### Example 11

Rapamycin was loaded at 30% into a dispersing media that consisted of 70% fish oil and 30% vitamin E. 1.5 grams of this formulation were placed into a vial and cryo-processed using a Spex Certiprep cryomill. The sample was processed for 8 cycles with cooling times of two minutes between cycles and a frequency of 20 impacts per second. The resulting material was warmed to room temperature, sheared and removed from the vial. The resulting formulation was tested on an Anton Paar MCR 301 rheometer and found to be highly viscous and shear thinning with small crystals evident throughout the sample. When a flow curve was conducted the viscosity was found to be 1.2 million Cps at 1 1/s (1 reciprocal second). A control formulation was also prepared where Rapamycin was loaded at 30% into a dispersing media that consisted of 70% fish oil and 30% vitamin E but not cryo processed. The control formulation was also tested on the MCR 301 rheometer and found to be very non homogeneous with drug crystals on the bottom and oil-based coating on top. The viscosity of this sample at 1 1/s as calculated through a flow curve was 1932 cps. The data clearly shows that cryoprocessing can be used to dramatically increase the viscosity and alter the rheology of coating materials.

### Example 12

When oil is used as the continuous phase, cryogrinding of selected solids generates a substantially-thickened reversible dispersion. What follows is rheology data obtained from Anton Paar, a supplier of rheometers. In these tests, Anton Paar used an MCR 301 series rheometer to quantify the shear rate dependent viscosity, the thixotropic behavior and recovery time, the structural behavior as a function of deformation and time, and for one set of samples the effect of temperature on the flow behavior. This information is useful input to our design of dispensing equipment since it shows that the material's viscosity is a function of temperature and shear history. (The Rapamycin prodrug is TAFA 93.)

TABLE C
RHEOLOGY SUMMARY

| | | Viscosity | | | Temperature |
|---|---|---|---|---|---|
| Sample | Base | (cP) | | Amplitude | Sweep |
| Description | Material | @ 1/sec | Rheology | Sweep | 18 to 50° C. |
| Coating only | 70:30 | 87 | Newtonian | G″ > G′ | |
| | FO:VitE | | | | |
| Coating only | 50:50 | 810 | Newtonian | G″ > G′ | |
| | FO:VitE | | | | |
| Coating only | 30:70 | 11,080 | Newtonian | G″ > G′ | |
| | FO:VitE | | | | |
| Rapamycin | 70:30 | 18,000 | shear thinning, | G″ > G′ | |
| insoluble | FO:VitE | | but at low shear | typical of | |
| (40%) | | | rates, viscosity | heterogeneous | |
| | | | increases with | material | |
| | | | increasing shear | | |
| Rapamycin | 50:50 | 20,000 | shear thinning | G″ > G′ | |
| soluble (50%) | FO:VitE | | | | |
| Rapamycin | 30:70 | 6,660,000 | shear thinning | G″ > G′ | Near linear |
| Pro-drug | FO:VitE | | | More fluid | decrease of |
| cryoground | | | | than solid | complex |
| (29.43%) | | | | ~10% strain, | viscosity |
| | | | | structure | through range |
| | | | | breaks down | |
| Rapamycin | 70:30 | 18,997,000 | Highly shear | G′ > G″, then | Complex |
| Pro-drug | FO:VitE | | thinning | G″ > G′ | viscosity |
| cryoground | | | | More solid | mostly |
| (29.63%) | | | | than fluid | independent |
| | | | | ~0.2% | of |
| | | | | strain, | temperature in |
| | | | | structure | this range |
| | | | | breaks down | |
| Rapamycin | 30:70 | 1,140 | Newtonian | G″ > G′ | Near linear |
| Pro-drug | FO:VitE | | | More fluid | decrease of |
| soluble | | | | than solid | complex |
| (10.19%) | | | | | viscosity |
| | | | | | through range |
| Rapamycin | 30:70 | 202,000 | Newtonian, | G″ > G′ | Near linear |
| Pro-drug | FO:VitE | | some shear | More fluid | decrease of |
| soluble | | | thinning at | than solid | complex |
| (25.49%) | | | higher rates | | viscosity |
| | | | | | through range |

G′ = storage modulus - refers to the tendency of the system to store energy
G″ = loss modulus - refers to the tendency of the system to dissipate energy

### Example 13

Cryogrinding in an oil medium can reduce or eliminate the need for strict environmental control over humidity when processing materials which are prone to hydrolysis. To demonstrate this, Rapamycin pro-drug was cryoground at 60% relative humidity and showed a shift in the OH band of the FTIR spectra indicating possible hydrolysis of the drug. Under low humidity (6%) this hydrolysis did not occur. When the Rapamycin pro-drug was ground at 60% relative humidity in a 70:30 Fish Oil:vitamin E formulation, hydrolysis did not occur.

### Example 14

As compared to formulations prepared where the drug is solubilized in a fish oil:vitamin E continuous phase, cryoground formulations exhibit extended release. For example, Rapamycin pro-drug dispersed in fish oil:vitamin E (25% drug, maximum solubility) completed elution in PBS in 9 days. However, Rapamycin pro-drug cryoground in fish oil:Vitamin E (34% drug), continued eluting for 17 days.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention, and exclusive use of all modifications that come within the scope of the appended claims is reserved. It is intended that the present invention be limited only to the extent required by the appended claims and the applicable rules of law.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

While the present inventions have been described in conjunction with various embodiments and examples, it is not intended that the present teachings be limited to such embodiments or examples. On the contrary, the present inventions encompass various alternatives, modifications, and equivalents, as will be appreciated by those of skill in the art.

The claims should not be read as limited to the described order or elements unless stated to that effect. It should be understood that various changes in form and detail may be made without departing from the scope of the appended claims. Therefore, all embodiments that come within the scope and spirit of the following claims and equivalents thereto are claimed.

1. A substantially-thickened therapeutic formulation comprising an oil-based composition and a therapeutic agent, wherein the therapeutic agent is of a reduced particle size.

2. The substantially-thickened therapeutic formulation of claim 1, wherein the particle size of the therapeutic agent has been reduced using cryogrinding.

3. The substantially-thickened formulation of claim 1, wherein the therapeutic agent is at a loading concentration that is higher than the maximum solubility concentration of the agent in the oil-based composition before cryogrinding.

4. The substantially-thickened formulation of claim 3, wherein the therapeutic agent is at a loading concentration of greater than 30% when the solubility of the agent in the oil-based composition before cryogrinding is less than 25%.

5. The substantially-thickened formulation of claim 3, wherein the therapeutic agent is at a loading concentration of 20%-50%.

6. The substantially-thickened formulation of claim 1, wherein the therapeutic agent is uniformly dispersed throughout the formulation, such that there is no bulk phase separation between the oil-based composition and the therapeutic agent.

7. The substantially-thickened formulation of claim 1, wherein the oil-based composition comprises an oil containing at least one lipid or fatty acid.

8. The substantially-thickened formulation of claim 7, wherein the fatty acid comprises one or more of arachidic acid, gadoleic acid, arachidonic acid, eicosapentaenoic acid (EPA), docosahexaenoic acid (DHA), butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, analogs and pharmaceutically acceptable salts thereof.

9. The substantially-thickened formulation of claim 1, wherein the oil-based composition comprises at least one or more of a fish oil, a biological oil, or vegetable oil.

10. The substantially-thickened formulation of claim 9, wherein the oil-based composition further comprises a vitamin E compound selected from the group consisting of alpha-tocopherol, beta-tocopherol, delta-tocopherol, gamma-tocopherol, alpha-tocotrienol, beta-tocotrienol, delta-tocotrienol, gamma-tocotrienol, alpha-tocopherol acetate, beta-tocopherol acetate, gamma-tocopherol acetate, delta-tocopherol acetate, alpha-tocotrienol acetate, beta-tocotrienol acetate, delta-tocotrienol acetate, gamma-tocotrienol acetate, alpha-tocopherol succinate, beta-tocopherol succinate, gamma-tocopherol succinate, delta-tocopherol succinate, alpha-tocotrienol succinate, beta-tocotrienol succinate, delta-tocotrienol succinate, gamma-tocotrienol succinate, vitamin E TPGS, mixed tocopherols, derivatives, analogs and pharmaceutically acceptable salts thereof.

11. The substantially-thickened formulation of claim 9, wherein the oil-based composition comprises a combination of a fish oil and vitamin E or an analog of vitamin E.

12. The substantially-thickened formulation of claim 1, wherein the therapeutic agent comprises one or more of an antioxidant, an anti-inflammatory agent, an anti-coagulant agent, a drug to alter lipid metabolism, an anti-proliferative, an anti-neoplastic, a tissue growth stimulant, a functional protein/factor delivery agent, an anti-infective agent, an imaging agent, an anesthetic agent, a chemotherapeutic agent, a tissue absorption enhancer, an anti-adhesion agent, a germicide, an analgesic, an antiseptic, or pharmaceutically acceptable salts, esters, analogues, derivatives, isomers, or prodrugs thereof.

13. The substantially-thickened formulation of claim 12, wherein the therapeutic agent is a Rapamycin derivative, a Rapamycin prodrug, a calcineurin inhibitor, an anti-proliferative, an anti-oxidant, an anti-neoplastic, vitamin E, or an analog of vitamin E, fructose, fish oil, or cetyl alcohol.

14. The substantially-thickened formulation of claim 12, wherein the therapeutic agent is selected from the group consisting of Rapamycin, Marcaine, Paclitaxel, Cyclosporine, Voclosporine, and Rifampicin.

15. The substantially-thickened formulation of claim 12, wherein the therapeutic agent is in a prodrug form.

16. The substantially-thickened formulation of claim 15, wherein, the therapeutic agent is a Rapamycin prodrug, and, after fragmentation, the Rapamycin prodrug particles have a distribution of size of about 1-15 μm (v,0.1), 16-35 μm (v,0.5), and 36-50 μm (v,0.9).

17. A method for forming a substantially-thickened formulation comprising:
(a) associating a composition comprising at least an oil-based composition and a therapeutic agent with a cryogenic liquid;
(b) fragmenting the composition;
(c) returning the composition to an ambient temperature, and
(d) optionally shearing the formulation such that the substantially-thickened formulation is formed.

18. The method of claim 17, wherein the step of associating the composition with a cryogenic liquid comprises at least one of suspending, submerging, surrounding, and cooling the composition with the cryogenic liquid.

19. The method of claim 17, wherein the therapeutic agent comprises one or more of an antioxidant, an anti-inflammatory agent, an anti-coagulant agent, a drug to alter lipid metabolism, an anti-proliferative, an anti-neoplastic, a tissue growth stimulant, a functional protein/factor delivery agent, an anti-infective agent, an imaging agent, an anesthetic agent, a chemotherapeutic agent, a tissue absorption enhancer, an anti-adhesion agent, a germicide, an analgesic, an antiseptic, or pharmaceutically acceptable salts, esters, or prodrugs thereof.

20. The method of claim 17, wherein the cryogenic liquid comprises liquid nitrogen.

21. The method of claim 17, wherein the composition is fragmented using at least one or more of sonication, grinding, impacting, shearing, shocking, shattering, granulating, pulverizing, shredding, crushing, homogenizing, milling, vibrating, vortexing, and shaking.

22. The method of claim 17, wherein the method further comprises the step of shearing the composition after the composition is processed.

23. The method of claim 17, wherein the oil-based composition is a fish oil.

24. The substantially-thickened formulation produced by the method of claim 17.

25. A method for forming a substantially-thickened formulation comprising:
(a) associating a composition comprising a therapeutic agent and a solvent with a cryogenic liquid, wherein the solvent does not dissolve the therapeutic agent;
(b) fragmenting the composition;
(c) optionally removing the solvent;
(d) associating the composition with an oil;
(e) associating the oil-based composition with a cryogenic liquid;
(e) fragmenting the oil-based/cryogenic liquid composition;
(f) returning the composition to an ambient temperature, and
(g) optionally shearing the composition such that a thickened formulation is formed.

26. The method of claim 25, wherein the solvent is hexane.

27. The method of claim 25, wherein the therapeutic agent is a Rapamycin prodrug.

28. The method of claim 27, wherein, after fragmentation, the Rapamycin prodrug particles have a distribution of size of about 0.1-10 μm (v,0.1), 11-30 μm (v,0.5), and 31-50 μm (v,0.9).

29. The method of claim 25, wherein the oil-based composition is a fish oil.

30. The method of claim 26, wherein the Rapamycin prodrug particles have a diameter of less than about 10 μm (v,0.9).

31. The method of claim 25, wherein the composition is sheared such that a thickened formulation is formed.

32. The substantially-thickened formulation produced by the method of claim 25.

33. A medical device, comprising:
a medical device structure; and
a coating formed on at least a portion of the medical device structure;
wherein the coating comprises a substantially-thickened therapeutic formulation, wherein the substantially-thickened therapeutic formulation comprises an oil-based composition and a therapeutic agent, wherein the therapeutic agent has been reduced by cryogrinding techniques to a solid of a reduced particle size.

34. The medical device of claim 33, wherein the therapeutic agent is at a loading concentration that is higher than the maximum solubility concentration of the agent in the oil before cryogrinding.

35. The medical device of claim 33, wherein the medical device is a vascular graft, hernia mesh, thin film, or stent.
